(19) United States
(12) Patent Application Publication
(10) Pub. No.: US 2017/0188964 A1
(43) Pub. Date: Jul. 6, 2017

(54) PHYSIOLOGICAL MONITORING SYSTEM FEATURING FLOORMAT AND HANDHELD SENSOR

(71) Applicant: TOSENSE, INC., La Jolla, CA (US)

(72) Inventors: Matthew BANET, San Diego, CA (US); Marshal Singh DHILLON, San Diego, CA (US); Susan Meeks PEDE, Encinitas, CA (US); Lauren Nicole Miller HAYWARD, San Diego, CA (US); Arthur DEPTALA, Santee, CA (US); Jonas Dean COCHRAN, Santee, CA (US)

(21) Appl. No.: 14/988,716

(22) Filed: Jan. 5, 2016

(51) Int. Cl. A61B 5/00; A61B 5/024; A61B 5/1455; A61B 5/0205; A61B 5/0452; A61B 5/0404

(52) U.S. Cl. CPC A61B 5/6892; A61B 5/0452; A61B 5/0404; A61B 5/14551; A61B 5/0205; A61B 5/4869; A61B 5/4875; A61B 5/02427

(57) ABSTRACT

The invention described herein is a system that features a Floormat and Handheld Sensor that operate in concert with a user's mobile device. The Floormat resembles a conventional bathroom scale, but features an enhanced set of measurements that include pulse rate and/or heart rate, SpO2, respiratory rate, weight, body composition, and Fluids. The Handheld Sensor features an integrated form factor that fits in a user's hand, which measures parameters such as blood pressure (e.g. systolic, diastolic, mean and pulse pressures), stroke volume, and cardiac output. Measurements of stroke volume and cardiac output require information from the Floormat (e.g., weight and body composition) to be sent to and processed by the Handheld Sensor. The Handheld Sensor can also make redundant measurements of heart rate, SpO2, and respiratory rate. Both systems transmit information through a wireless interface to a web-based system, where a clinician can analyze it to help diagnose a user.

### BACKGROUND AND FIELD OF THE INVENTION

1. Field of the Invention

The invention relates to sensors that measure physiological signals from a user (e.g. a patient), and the use of such sensors.

2. General Background

Physiological sensors, such as vital sign monitors, typically measure signals from a patient to determine time-varying waveforms, e.g. thoracic bio-impedance (TBI), bio-reactance (BR), and electrocardiogram (ECG) waveforms, with electrodes that attach to the patient's skin. These waveforms can be processed/analyzed to extract other medically relevant parameters such as heart rate (HR) and heart rate variability (HRV), respiration rate (RR), stroke volume (SV), cardiac output (CO), and information relating to thoracic fluids, e.g. thoracic fluid index (TFC) and general body fluids (Fluids). Certain physiological conditions can be identified from these parameters using one-time measurements; other conditions require observation of time-dependent trends in the parameters in order to identify the underlying condition. In all cases, it is important to measure the parameters with high repeatability and accuracy.

Some conditions require various physiological parameters to be measured over a relatively short period of time in order to identify the condition. For example, Holter monitors can characterize various types of cardiac arrhythmias by measuring HR, HRV, and ECG waveforms over periods ranging from a day to a few weeks. On the other hand, chronic diseases such as congestive heart failure (CHF) and end-stage renal disease (ESRD) typically require periodic measurements of Fluids and weight throughout the patient's life in order to identify the condition. Not surprisingly, patient compliance with measurement routines typically decreases as the measurement period increases. This is particularly true when measurements are made outside of a conventional medical facility, e.g., at the patient's home or in a residential facility such as a nursing home.

Furthermore, the measured values of some physiological parameters will vary with the location at which the parameters are measured, while those associated with other physiological parameters are relatively independent of the location at which the parameters are measured. For example, parameters such as HR, which depends on the time-dependent variation of R-R intervals associated with QRS complexes in ECG waveforms, are relatively insensitive to sensor positioning. Likewise, pulse oximetry (SpO2) and pulse rate (PR), as measured from photoplethysmogram (PPG) waveforms with a pulse oximeter, show little variance with measurement location.

On the other hand, measurements that depend on amplitude-dependent features in waveforms, such as TFC or Fluids, will be strongly dependent on the measurement location, e.g. the positioning of electrodes. In the case of TFC, for example, the measured value depends strongly on the sensed impedance between a set of electrodes. And this, in turn, will vary with the electrodes' placement. TFC deviation in the day-to-day placement of the electrodes can result in measurement errors. This, in turn, can lead to misinformation (particularly when trends of the measured parameters are to be extracted), thereby nullifying the value of such measurements and thus negatively impacting treatment.

Like TFC, measured values of blood pressure (BP), such as systolic (SYS), diastolic (DIA), and pulse (PP) pressures are typically sensitive to the location at which the parameter is measured. For example, blood pressure measured at the brachial artery with a sphygmomanometer (i.e. a manual blood pressure cuff) or with an oscillometric device (i.e. an automated blood pressure cuff measuring oscillometric waveforms) will typically be different from that measured at other locations on the body, such as the wrist, thigh, finger, or even the opposite arm. Mean arterial pressure (MAP) is less sensitive to position, as it is relatively constant throughout the body. Body (e.g. skin) temperature is similarly dependent on the location at which it is measured, although core temperature (TEMP), as measured from the ear or mouth, is relatively consistent.

3. Sensors, Devices, and Relevant Physiology

Disposable electrodes that measure ECG and TBI waveforms are typically worn on the patient's chest or legs and include: i) a conductive hydrogel that contacts the patient's skin; ii) a Ag/AgCl-coated eyelet that contacts the hydrogel; iii) a conductive metal post that connects to a lead wire or cable extending from the sensing device; and iv) an adhesive backing that adheres the electrode to the patient. Unfortunately, during a measurement, the lead wires can pull on the electrodes if the device is moved relative to the patient's body, or if the patient ambulates and snags the lead wires on surrounding objects. Such pulling can be uncomfortable or even painful, particularly where the electrodes are attached to hirsute parts of the body, and this can inhibit patient compliance with long-term monitoring. Moreover, these actions can degrade or even completely eliminate adhesion of the electrodes to the patient's skin, and in some cases completely destroy the electrodes' ability to sense the physiological signals at various electrode locations.

Some devices that measure ECG and TBI waveforms are worn entirely on the patient's body. These devices have been developed to feature simple, patch-type systems that include both analog and digital electronics connected directly to underlying electrodes. Such devices, like the Holter monitors described above, are typically prescribed for relatively short periods of time, e.g. for a period of time ranging from a day to several weeks. They are typically wireless and include features such as Bluetooth® transceivers to transmit information over a short distance to a second device, which then transmits the information via a cellular radio to a web-based system.

SpO2 values are almost always measured at the patient's fingers, earlobes, or, in some cases, the forehead. In these cases, patients wear an optical sensor to measure PPG waveforms, which are then processed to yield SpO2 and PR values. TEMP is typically measured with a thermometer inserted into the patient's mouth, or with an optical sensor featuring an infrared-sensitive photodiode pointed into the patient's ear.

Assessing Fluids, TFC, weight, and hydration status is important in the diagnosis and management of many diseases. For example, ESRD occurs when a patient's kidneys are no longer able to work at a level needed for day-to-day life. The disease is most commonly caused by diabetes and high blood pressure, and is characterized by swings in SYS and DIA along with a gradual increase in Fluids throughout the body. Patients suffering from ESRD typically require hemodialysis or ultrafiltration to remove excess Fluids. Thus, accurate measurement of this parameter and/or TFC to characterize ESRD can eliminate the need for empirical clinical estimations that often lead to over-removal or under-removal of fluids during dialysis, thereby preventing hemodynamic instability and hypotensive episodes (Anand et al., “Monitoring Changes in Fluid Status With a Wireless Multisensor Monitor: Results From the Fluid Removal During Adherent Renal Monitoring (FARM) Study,” Congest Heart Fail. 2012; 18:32-36). A similar situation exists with respect to CHF, which is a complicated disease typically monitored using a “constellation” of physiological factors, e.g., fluid status (e.g. Fluids, TFC), vital signs (i.e., HR, RR, TEMP, SYS, DIA, and SpO2), and hemodynamic parameters (e.g. CO, SV). Accurate measurement of these parameters can aid in managing patients, particularly in connection with dispensing diuretic medications, and thus reduce expensive hospital readmissions (Packer et al., “Utility of Impedance Cardiography for the Identification of Short-Term Risk of Clinical Decompensation in Stable Patients With Chronic Heart Failure,” J Am Coll Cardiol 2006; 47:2245-52).

CHF is a particular type of heart failure (HF), which is a chronic disease driven by complex pathophysiology. In general terms, HF occurs when SV and CO are insufficient to adequately perfuse the kidneys and lungs. Causes of this disease are well known and typically include coronary heart disease, diabetes, hypertension, obesity, smoking, and valvular heart disease. In systolic HF, ejection fraction (EF) can be diminished (<50%), whereas in diastolic HF this parameter is typically normal (>65%). The common signifying characteristic of both forms of heart failure is time-dependent elevation of the pressure within the left atrium at the end of its contraction cycle, or left ventricular end-diastolic pressure (LVEDP). Chronic elevation of LVEDP causes transudation of fluid from the pulmonary veins into the lungs, resulting in shortness of breath (dyspnea), rapid breathing (tachypnea), and fatigue with exertion due to the mismatch of oxygen delivery and oxygen demand throughout the body. Thus, early compensatory mechanisms for HF that can be detected fairly easily include increased RR and HR.

As CO is compromised, the kidneys respond with decreased filtration capability, thus driving retention of sodium and water and leading to an increase in intravascular volume. As the LVEDP rises, pulmonary venous congestion worsens. Body weight increases incrementally, and fluids may shift into the lower extremities. Medications for HF are designed to interrupt the kidneys' hormonal responses to diminished perfusion, and they also work to help excrete excess sodium and water from the body. However, an extremely delicate balance between these two biological treatment modalities needs to be maintained, since an increase in blood pressure (which relates to afterload) or fluid retention (which relates to preload), or a significant change in heart rate due to a tachyarrhythmia, can lead to decompensated HF. Unfortunately, this condition is often unresponsive to oral medications. In that situation, admission to a hospital is often necessary for intravenous diuretic therapy.

In medical centers, HF is typically detected using Doppler/ultrasound, which measures parameters such as SV, CO, and EF. In the home environment, on the other hand, gradual weight gain measured with a simple weight scale is likely the most common method used to identify CHF. However, by itself, this parameter is typically not sensitive enough to detect the early onset of CHF—a particularly important stage when the condition may be ameliorated simply and effectively by a change in medication or diet.

SV is the mathematical difference between left ventricular end-diastolic volume (EDV) and end-systolic volume (ESV), and represents the volume of blood ejected by the left ventricle with each heartbeat; a typical value is about 70-100 mL. CO is the average, time-dependent volume of blood ejected from the left ventricle into the aorta and, informally, indicates how efficiently a patient's heart pumps blood through their arterial tree; a typical value is about 5-7 L/min. CO is the product of HR and SV.

CHF patients—particular those suffering from systolic HF—may receive implanted devices such as pacemakers and/or cardioverter-defibrillators to increase EF and subsequent blood flow throughout the body. These devices may include circuitry and algorithms to measure the electrical impedance between different leads of the device. Some implanted devices process this impedance to calculate a “fluid index”. As thoracic fluid increases in the CHF patient, the impedance typically is reduced, and the fluid index increases.

4. Clinical Solutions

Many of the above-mentioned parameters can be used as early markers or indicators that signal the onset of CHF. EF is typically low in patients suffering from this chronic disease, and it can be further diminished by factors such as a change in physiology, an increase in sodium in the patient's diet, or non-compliance with medications. This is manifested by a gradual decrease in SV, CO, and SYS that typically occurs between two and three weeks before hospitalization becomes necessary to treat the condition. As noted above, the reduction in SV and CO diminishes perfusion to the kidneys. These organs then respond with a reduction in their filtering capacity, thus causing the patient to retain sodium and water and leading to an increase in intravascular volume. This, in turn, leads to congestion, which is manifested to some extent by a build-up of fluids in the patient's thoracic cavity (e.g. TFC). Typically, a detectable increase in TFC occurs about 1-2 weeks before hospitalization becomes necessary. Body weight increases after this event (typically by between three and five pounds), thus causing fluids to shift into the lower extremities. At this point, the patient may experience an increase in both HR and RR to increase perfusion. Nausea, dyspnea, and weight gain typically grow more pronounced a few days before hospitalization becomes necessary. As noted above, a characteristic of decompensated HF is that it is often unresponsive to oral medications; thus, at this point, intravenous diuretic therapy in a hospital setting often becomes mandatory. A hospital stay for intravenous diuretic therapy typically lasts about 4 days (costing several thousands of dollars per day, or more), after which the patient is discharged and the above-described cycle may start over once again.

Such cyclical pathology and treatment is physically taxing on the patient, and economically taxing on society. In this regard, CHF and ESRD affect, respectively, about 5.3 million and 3 million Americans, resulting in annual healthcare costs estimated at $45 billion for CHF and $35 billion for ESRD. CHF patients account for approximately 43% of annual Medicare expenditures, which is more than the combined expenditures for all types of cancer. Somewhat disconcertingly, roughly $17 billion of this is attributed to hospital readmissions. CHF is also the leading cause of mortality for patients with ESRD, and this demographic costs Medicare nearly $90,000/patient annually. Thus, there understandably exists a profound financial incentive to keep patients suffering from these diseases out of the hospital. Starting in 2012, U.S. hospitals have been penalized for above-normal readmission rates. Currently, the penalty has a cap of 1% of payments, growing to over 3% in the next 3 years.

Of some promise, however, is the fact that CHF-related hospital readmissions can be reduced when clinicians have access to detailed information that allows them to remotely titrate medications, monitor diet, and promote exercise. In fact, Medicare has estimated that 75% of all patients with ESRD and/or CHF could potentially avoid hospital readmissions if treated by simple, effective programs.

Thus, in order to identify precursors to conditions such as CHF and ESRD, physicians can prescribe physiological monitoring regimens to patients living at home. Typically, such regimens require the use of multiple standard medical devices, e.g. blood pressure cuffs, weight scales, and pulse oximeters. In certain cases, patients use these devices daily and in a sequential manner, i.e., one device at a time. The patient then calls a central call center to relay their measured parameters to the call center. In more advanced systems, the devices are still used in a sequential manner, but they automatically connect through a short-range wireless link (e.g. a Bluetooth® system) to a “hub,” which then forwards the information to a call center. Often, the hub features a simple user interface that presents basic questions to the patient, e.g. questions concerning their diet, how they are feeling, and whether or not medications were taken.

Ultimately, however, and regardless of how sophisticated such instrumentation may be, in order for such monitoring to be therapeutically effective, it is important for the patient to use their equipment consistently, both in terms of the duration and manner in which it is used. Less-than-satisfactory consistency with the use of any medical device (in terms of duration and/or methodology) may be particularly likely in an environment such as the patient's home or a nursing home, where direct supervision may be less than optimal.

### SUMMARY OF THE INVENTION

In view of the foregoing, it would be beneficial to provide a monitoring system that is suitable for home use. Particularly valuable would be a system that is wireless and conveniently measures a collection of vital signs and hemodynamic parameters. Ideally, such a system would be easy to use and feature a simple form factor that integrates into the user's day-to-day activities. The monitoring system according to the invention, which facilitates monitoring a user for HF, CHF, ESRD, cardiac arrhythmias, and other diseases, is designed to achieve this goal.

The invention described herein is a system that features a Floormat and Handheld Sensor that operate in concert with the user's mobile device. The Floormat resembles a conventional bathroom scale, but features an enhanced set of measurements that include PR and/or HR, SpO2, RR, weight, body composition, and Fluids. The Handheld Sensor features an integrated form factor that fits in a user's hand, which measures parameters such as BP (SYS, MAP, and DIA), TFC, SV, and CO. Measurements of SV and CO require information from the Floormat (e.g., weight and body composition). The Handheld Sensor can also make redundant measurements of HR, SpO2, and RR. Both systems transmit information through a wireless interface to a web-based system, where a clinician can analyze it to help diagnose a user.

The invention is based in part on the discovery that the bio-impedance signals (e.g. TBI and/or BR waveforms) used to determine vital signs and hemodynamic parameters can be measured over a conduction pathway that extends from the user's wrist to a location on their thoracic cavity, e.g. their chest or belly button. The form factor of the Handheld Sensor described herein accommodates such measurements with a system that is comfortable, easy to use, and includes re-usable electrode materials to reduce costs. Measurements made by the Handheld Sensor use the belly button as a ‘fiducial’ marker, as described in detail below. This location, which is present on nearly all users, facilitates consistent, daily measurements that reduce errors due to positioning that normally impact impedance measurements. In this and other ways, the Handheld Sensor provides an effective tool for characterizing users with chronic diseases, such as CHF, ESRD, and hypertension.

While a specific embodiment of the Handheld Sensor is described herein, this system can also take on other form factors. These include a watch, wristband, sling, and other systems designed to be worn on or near the hand and wrist.

In one aspect, the invention provides a system for monitoring SV from a user. The system features: 1) a wireless Floormat configured to rest on a substantially horizontal surface (e.g. a floor) that includes a weight-measuring system with at least one load cell and an amplifier system that measures a voltage from the load cell, and a algorithm that analyzes the voltage to determine a weight value; and 2) a wireless Handheld Sensor that is held in the user's hand or wrist while being pressed against a second portion of their body (e.g. belly, torso) while the user stands on the Floormat.

The Handheld Sensor features an impedance-measuring system with an electrode configuration that measures a set of analog impedance values that are digitized and processed as described below to form a TBI or BR waveform. A processing system that can be part of the Floormat, Handheld Sensor, or external wireless device receives the weight value from the Floormat and the TBI or BR waveforms from the Handheld Sensor, and then processes this information to calculate SV.

In embodiments, the impedance-measuring system features four electrodes, with two electrodes positioned on a wrist-mounted component (which includes a watch or related form factors, as described above), and two electrodes positioned on an exposed surface of the Handheld Sensor (e.g. a bottom surface) that can be brought in contact with another portion of the user's body (e.g. the belly) when the user holds it. The electrodes measure bio-impedance and bio-reactance signals as is described in detail below, and use these to generate TBI and BR waveforms.

In another aspect, the invention includes a similar system featuring both a Floormat and Handheld Sensor, again working in concert. Here, the processing system (which, again, may be part of either of these components, or alternatively part of an external device, such as a mobile device) receives the weight value and uses it and impedance signals from the Handheld Sensor to calculate SV as described above. The processing system also calculates a value of Fluids for the user from impedance signals measured by both the Floormat and Handheld Sensor. The value of Fluids is calculated from a combination of DC components of TBI and BR waveforms measured by these sensors, wherein the combination can be, for example, an average, weighted average, or summation of these values.

In another aspect, the invention provides a system for monitoring a user suffering from HF (e.g. CHF). Similar to that described above, the invention includes a Floormat that measures Fluids from the user's lower extremities, weight, and SpO2 from the toe, and a Handheld Sensor that receives the weight value and uses it and impedance signals to measure SV. The processing system receives these parameters, and further processes them to detect trends that may indicate the onset of HF. For example, the processing system can be configured to indicate an alarm when a trend in any of these values exceeds a first predetermined threshold value (as is the case for weight and Fluids during episodes of CHF), or falls below a predetermined threshold value (as is the case for SpO2, SV, and CO).

In another aspect, the invention provides a system for monitoring a ballistocardiogram (BCG) signal from a user. The invention features a Floormat, similar to that described above, that includes one or more load cells that measure a time-dependent voltage waveform when the user stands on it. Simultaneously, the Handheld Sensor measures a time-dependent PPG waveform from the user's fingers (alternatively, this waveform can be measured from the feet by an optical system within the Floormat). A processing system receives the time-dependent voltage waveform from the Floormat and the time-dependent PPG waveform from the Handheld Sensor. It analyzes a first set of pulses in the time-dependent PPG to determine a set of fiducial markers, and then analyzes the set of fiducial markers to average together multiple sections of the time dependent voltage waveform to determine the BCG signal from a user.

In embodiments, the weight-measuring system includes an electrical amplifier system configured to isolate and digitize an AC signal from the time-dependent voltage waveform (BCG-AC). Computer code in the processing system then collectively analyzes the BCG-AC signal and a digital version of the time-dependent PPG waveform (PPG signal). For example, the code can processes a heartbeat-induced pulse within the PPG signal (e.g. the maximum value of the pulse or its derivative) to determine a fiducial marker to analyze the BCG-AC signal, and then use this marker to detect a set of waveform segments within the BCG-AC signal that temporally follow the marker. Once the set of waveform segments are isolated, they can be averaged together to form a single BCG pulse having a relatively good signal-to-noise ratio. This technique is referred to herein as ‘beatstacking’. The beatstacked BCG pulse can then be used to determine a physiological parameter, e.g. a pulse transit time or a BP value calculated therefrom.

In yet another aspect, the invention provides a system for monitoring a pulse transit time from a user. The system calculates the transit time from time-dependent waveforms measured by both the Floormat (e.g. PPG or BCG waveforms) and the Handheld Sensor (PPG, ECG, TBI, and/or BR waveforms). Once calculated, the transit time can be used to calculate, e.g., a BP value.

Typically the Floormat and Handheld Sensor both include paired wireless transmitters for sending and receiving information. In preferred embodiments the transmitters are based on Bluetooth® or 802.11, although other short and long-range wireless systems can be used.

The Handheld Sensor features an electrical impedance system having at least four electrodes, at least one of which is configured to inject an electrical current into the user's body, and at least one of which is configured to measure a signal induced by the electrical current and representative of a TBI and/or BR waveform. A second wireless system within the sensor receives the weight or other SV calibration from the Floormat. An internal processing system receives signals from the electrical impedance system and converts them into a set of impedance values, and then analyzes the set of impedance values and the weight or SV calibration to calculate SV.

In embodiments, the SV calibration includes a value representing the user's weight and/or body composition. These values, along with data collected using known measurements of SV, are used to calculate a volume conductor, described in more detail below.

In embodiments, the processing system features computer code configured to analyze the set of impedance values to determine the SV. For example, the computer code can calculate a derivative of the set of impedance values to determine a dΔZ(t)/dt waveform, from which it calculates a maximum value or an area of a pulse therein. The computer code can also analyze the dΔZ(t)/dt waveform to determine an ejection time or a baseline impedance (Z₀) value. The computer code can then process these values to determine SV using Eq. 1:

$$SV \sim \frac{{( d Δ Z ( t ) / dt )}_{max}}{{Z}_{o}} LVET \qquad ( 1 )$$

or, alternatively:

$$SV \sim \sqrt{\frac{{( d Δ Z ( t ) / dt )}_{max}}{{Z}_{o}}} LVET \qquad ( 2 )$$

In embodiments, the Handheld Sensor receives a weight value from the Floormat. Alternatively it can receive this value from another source, e.g. a Bluetooth®-enabled scale, or through manual entry using a software application and mobile device. The processing system can then use the weight to determine SV from the equation:

$$SV = {V}_{c} \frac{{( d Δ Z ( t ) / dt )}_{max}}{{Z}_{o}} LVET \qquad ( 3 )$$

or, alternatively:

$$SV = {V}_{c} \sqrt{\frac{{( d Δ Z ( t ) / dt )}_{max}}{{Z}_{o}}} LVET \qquad ( 4 )$$

where V_{c} is a volume conductor calculated from the value of weight and/or body composition. In some cases, V_{c} is determined from weight and a calibration factor determined from measurements (taken, e.g., during a clinical study) that include a known value for SV.

In still other aspects, the system calculates CO by also measuring HR as described below (e.g. using an ECG waveform), and then collectively processing SV and HR (e.g., by taking the product) to determine CO.

To determine a pulse transit time, the processing system features computer code configured to: i) calculate a mathematical derivative of the impedance values to determine a set of derivative values; and ii) determine a local maximum of the set of derivative values to determine the first pulsatile component; and/or iii) determine a zero-point crossing of the set of derivative values to determine the first pulsatile component. The computer code may also be configured to: i) estimate the set of derivative values with a mathematical function; and ii) analyze the mathematical function to determine the first pulsatile component.

In embodiments, the computer code is configured to determine a local maximum of cardiac rhythm values to determine the second pulsatile component, where the cardiac rhythm values are representative of an ECG waveform. For example, the computer code can be configured to determine a QRS complex (e.g. calculate the Q or R point) in the ECG waveform to determine the second pulsatile component. It can also further process the cardiac rhythm values to determine a HR value, e.g. by calculating a time interval separating the first and second R points.

The measurement system described herein has many advantages. Collectively, the Floormat and Handheld Sensor provide a single, easy-to-use system that a user can deploy to measure all their vital signs, complex hemodynamic parameters, and basic wellness-related parameters such as weight, percent body fat, and muscle mass. Ideally the system is used in much the same way as a conventional bathroom scale. Such ease of use may increase compliance, thereby motivating daily use. And with this, the measurement system can calculate trends in a user's physiological parameters, thereby allowing better detection of certain disease states and/or management of chronic conditions such as HF, CHF, diabetes, hypertension, chronic obstructive pulmonary disease (COPD), ESRD, and kidney failure.

Still other advantages should be apparent from the following detailed description, and from the claims.

### BRIEF DESCRIPTION OF THE DRAWINGS

FIG. 1 is a schematic, side view of a user being monitored by the system according to the invention, which includes a Floormat that the user stands on, a Handheld Sensor that the user holds in their hand and presses against their belly, and a mobile device that connects wirelessly to these components and to a web-based system;

FIG. 2 is a flow chart of an algorithm that the system of FIG. 1 uses to calculate SV;

FIG. 3 is a two-dimensional, top view of the Floormat of FIG. 1;

FIG. 4 is a three-dimensional, side view of the Floormat of FIG. 3;

FIG. 5A is a schematic, front view of a user showing the current conduction path deployed by the Handheld Sensor of FIG. 1;

FIG. 5B is a photograph of a user holding the Handheld Sensor against their belly;

FIG. 6A is a three-dimensional, side view of the Handheld. Sensor of FIG. 1 wherein the Sensor's internal components are covered with a mechanical housing;

FIG. 6B is a three-dimensional, side view of the Handheld Sensor of FIG. 6A wherein the Sensor's mechanical housing is shown as being transparent to indicate the Sensor's internal components;

FIG. 7A is a three-dimensional, top view of the handheld Sensor of FIG. 6A;

FIG. 7B is a photograph of the Handheld Sensor's electrodes, which include inflatable bladders covered with a fabric that is both stretchable and conductive, in an un-inflated state;

FIG. 7C is a photograph of the Handheld Sensor's electrodes in an inflated state;

FIG. 8 is a schematic drawing showing locations on the human body were the Handheld Sensor measures physiological waveforms having pulsatile components;

FIG. 9 is a plot of time-dependent ECG, ΔZ(t), PPG, d(ΔZ(t))/dt, and d(PPG)/dt waveforms measured with the Handheld Sensor; and

FIG. 10 is a table showing various physiological conditions and how they are predicted by trends in certain physiological parameters measured by the system of FIG. 1.

### DETAILED DESCRIPTION

### 1. System Overview

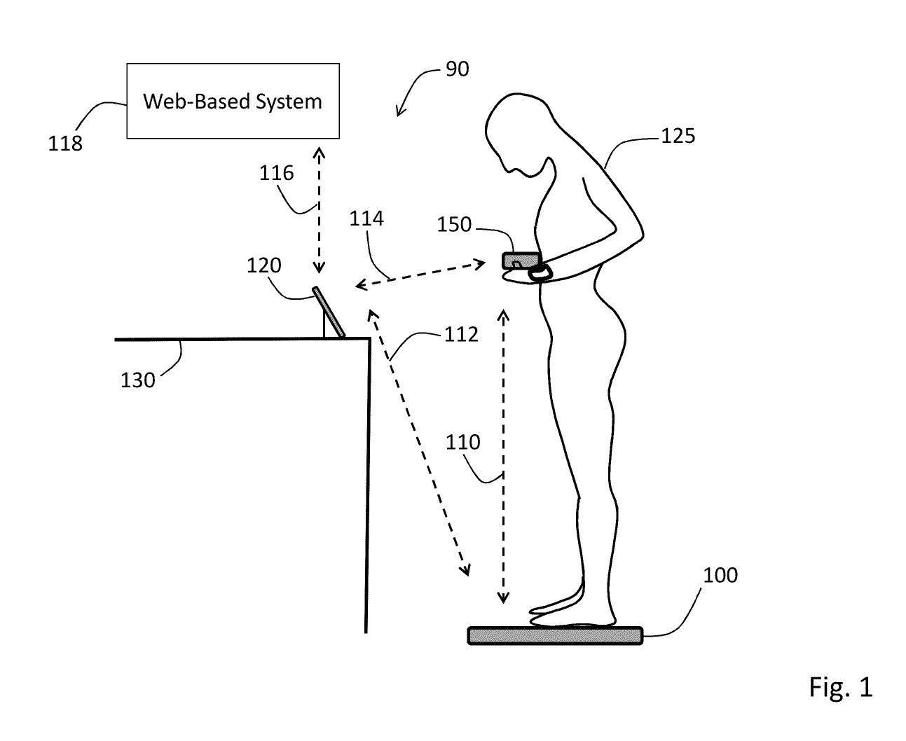

FIG. 1 shows a system 90 featuring a Floormat 100 and Handheld Sensor 150 working in concert to measure a user 125 according to the invention. Both the Floormat 100 and Handheld Sensor 150 feature a collection of physiological sensors, described in detail below, along with internal wireless devices that communicate with each other and an external mobile device 120. The goal of the system 90 is to quickly and non-invasively measure all five vital signs (HR, RR, SpO2, BP, and TEMP), hemodynamic parameters (SV, CO, TFC, Fluids), and biometric parameters (weight, body composition) with a collection of sensors that are easy-to-use, low-cost, inconspicuous, and seamlessly connect to the cloud. A rationale for the system 90 is that most disease states are predicted not by a single parameter (e.g. BP), but rather by a ‘constellation’ of parameters that may trend in different directions. However a complicating factor in monitoring such parameters is that they typically cannot be measured with a single device, or from a single location on the body. Thus, the system 90 is designed to measure all the above-described parameters using as few sensors as possible.

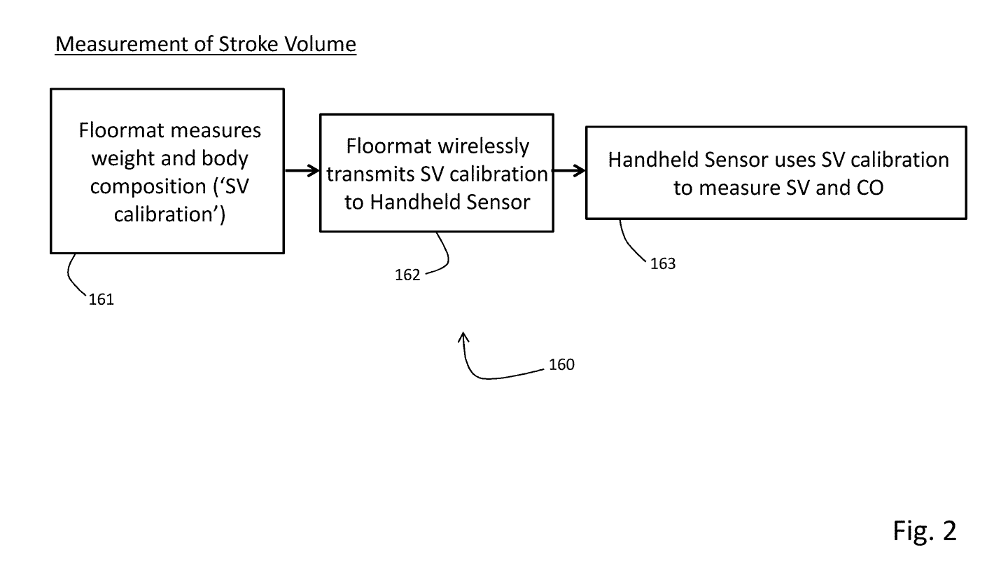

The Floormat 100 and Handheld Sensor 150 may each use parameters measured by the other device to complete a measurement. For example, the Handheld Sensor 150 may use weight or an SV calibration, as measured by the Floormat 100, to determine SV. Likewise, the Floormat 100 may use PPG waveforms, as measured by the Handheld Sensor, to perform beatstacking and measure BCG pulses. FIG. 2, as an example, indicates an algorithm 160 featuring a first step (161) wherein the Floormat 100 measures weight and body composition to determine an ‘SV calibration’. Using a second step (162) its internal wireless transmitter then wirelessly transmits the SV calibration to the Handheld Sensor 150, which finally uses a third step (163) to measure waveforms and collectively process the waveforms and SV calibration to determine SV and, ultimately, CO (CO is the product of SV and HR).

Each device in the system 90 transmits information to each other, as shown by arrow 110, and to the external mobile device 120, as shown by arrows 112, 114. The mobile device 120, for example, can be a cellular telephone or tablet computer using a customized software application (e.g. one running on Android or iOS platforms, and downloaded from the cloud). During a measurement, the mobile device 120 is typically placed on a horizontal surface 130, such as a bathroom countertop. Once it receives information, the mobile device 120 transmits it to a Web-based System 118 for follow-on analysis, e.g. by a clinician or a data-analytics software platform.

Collectively the Floormat 100 and Handheld Sensor 150 non-invasively measure all vital signs (e.g. HR, RR, BP, SpO2, and TEMP), hemodynamic parameters (SV, CO, TFC, and Fluids), and biometric parameters (weight, body composition). Ideally these parameters are measured at roughly the same time each day, for example in the morning before the user brushes their teeth or takes a shower. Hardware and software systems for making these measurements are described in detail below. In other embodiments, the Floormat 100 as described herein can be replaced by a similar device such as that described in the following co-pending application, the contents of which are incorporated herein by reference: ‘FLOORMAT PHYSIOLOGICAL SENSOR’, U.S.S.N FLOORMAT PHYSIOLOGICAL SENSOR (U.S. Ser. No. _{______}, Filed _{______}); COMBINED FLOORMAT AND BODY-WORN PHYSIOLOGICAL SENSORS (U.S. Ser. No. _{______}, Filed _{______}); HANDHELD PHYSIOLOGICAL SENSOR (U.S. Ser. No. _{______}, Filed _{______}); PHYSIOLOGICAL MONITORING SYSTEM FEATURING FLOORMAT AND HANDHELD SENSOR (U.S. Ser. No. _{______}, Filed _{______}); and PHYSIOLOGICAL MONITORING SYSTEM FEATURING FLOORMAT AND WIRED HANDHELD SENSOR.

### 2. Floormat

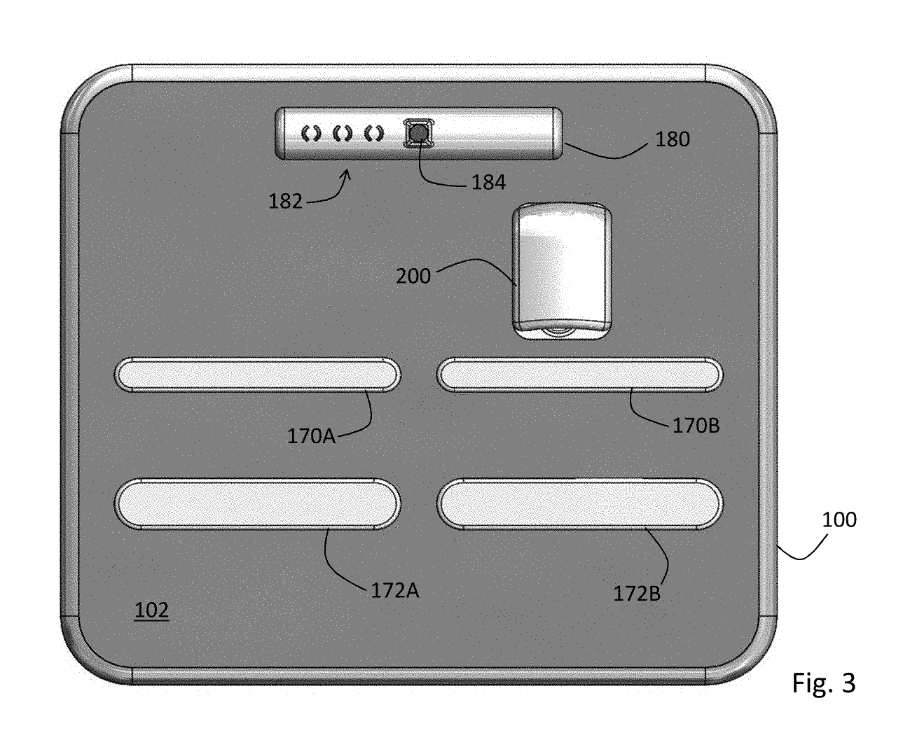

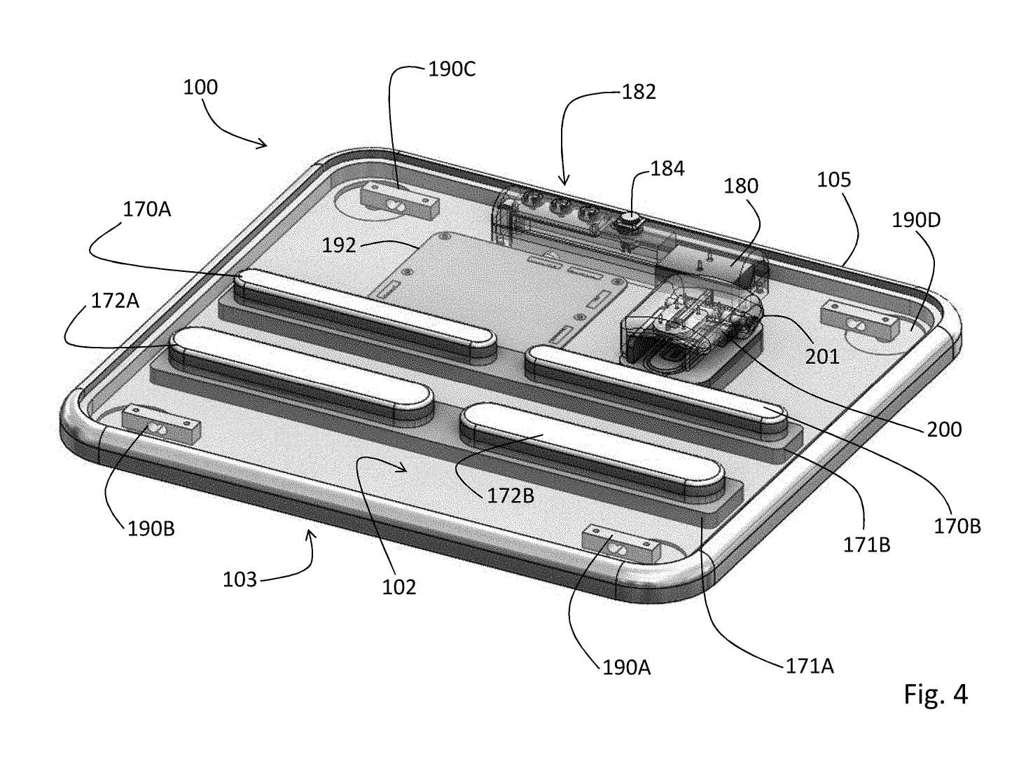

FIGS. 3 and 4 show, respectively, a top and three-dimensional view of a Floormat 100 according to the invention. In this embodiment, as described in more detail below, the Floormat 100 measures HR (along with HRV), SpO2, RR, Fluids, weight, and body composition. Resembling a conventional bathroom scale, the Floormat 100 features a top surface 102 and base 103 that are held together by a rigid border 105. Four load cells 190A-D, each connected to a respective corner of the base 103, sit on a horizontal surface (e.g. a floor) to support the Floormat. The load cells 190A-D are used to measure the user's weight and in some cases a BCG waveform, as is described in detail below.

A toe-clip sensor 200 designed to fit around a user's big toe measures HR, SpO2, and RR, as described in more detail below. The toe-clip sensor 200 is supported by the Floormat's top surface 102, and features a spring-loaded clip 201 that gently presses an optical system against the toe to maximize measurement performance. The optical system features radiation-emitting diodes (LEDs) operating near the red (630 nm) and infrared (905 nm) spectral regions, and a photodetector that receives radiation after it transmits through the user's toe. Such components are similar to those used on conventional medical devices for SpO2 measurements designed for the fingers, earlobes, and forehead. More specifically, a digital system within the Floormat's circuit board 192 processes the waveforms to determine SpO2. This measurement is described in more detail in the following co-pending patent applications, the contents of which are incorporated herein by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Feb. 19, 2014; and “BODY-WORN SENSOR FOR CHARACTERIZING PATIENTS WITH HEART FAILURE,” U.S. Ser. No. 14/145,253, filed Dec. 31, 2013, and ‘FLOORMAT PHYSIOLOGICAL SENSOR’, U.S.S.N FLOORMAT PHYSIOLOGICAL SENSOR (U.S. Ser. No. _{______}, Filed _{______}); COMBINED FLOORMAT AND BODY-WORN PHYSIOLOGICAL SENSORS (U.S. Ser. No. _{______}, Filed _{______}); HANDHELD PHYSIOLOGICAL SENSOR (U.S. Ser. No. _{______}, Filed _{______}); PHYSIOLOGICAL MONITORING SYSTEM FEATURING FLOORMAT AND HANDHELD SENSOR (U.S. Ser. No. _{______}, Filed _{______}); and PHYSIOLOGICAL MONITORING SYSTEM FEATURING FLOORMAT AND WIRED HANDHELD SENSOR. To summarize, during a measurement, the digital system on the circuit board 192 alternatively powers red and infrared LEDs; typically these are included within a dual-emitting LED. This process generates the two distinct PPG waveforms. Using both digital and analog filters, the digital system extracts AC and DC components from the red (RED(AC) and RED(DC)) and infrared (IR(AC) and IR(DC)) PPG waveforms, which it then processes to determine SpO2, as described in the above-referenced patent applications.

Algorithms operating on the Floormat's circuit board 192 additionally calculate RR from a low-frequency envelope that modulates the PPG waveform. The frequency of this envelope, and thus RR, can be determined using known techniques in the art, such as Fourier analysis or by simply counting the breathing-induced pulses therein. Typically measurements of RR take about 30 seconds, which is the time required for about 6-10 breaths. Measurements of HR and SpO2 are typically done faster, as these values can be determined with just a few heartbeats. In all cases, the system makes these and other measurements in parallel, as described in detail below.

The Floormat's top surface 102 also supports sets of electrodes 170A,B, 172A,B, which are secured with a pair of plastic arms 171A,B that hold them securely in place during a measurement. The electrodes 170A,B, 172A,B use impedance technologies to collectively measure time-dependent TBI and BR waveforms from which the microprocessor calculates impedance changes and Fluids, as described in more detail below. Such parameters are calculated from DC components of the TBI and BR waveforms. Parameters such as SV and CO, which are calculated from the AC components of the TBI and BR waveforms, are typically difficult to measure from the feet, and are thus are preferably measured with the Handheld Sensor 150, as described in more detail below. Typically the electrodes 170A,B, 172A,B are reusable components fabricated from conductive materials such as stainless steel or foam covered with a conductive fabric. Use of other electrode materials is also within the scope of this invention. To make a measurement, electrodes 170A,B inject high-frequency (100 kHz), low-amperage (<6 mA) current into, respectively, the user's left and right feet. Typically the current injected by the respective electrodes is 180° out of phase. Electrodes 172A,B sense bioelectric signals that vary with the impedance (e.g. electrical resistance) encountered by the injected current. Processing of the signals sensed by electrodes 172A,B yields time-dependent impedance parameters (TBI and BR waveforms, both measures of bio-impedance) that track amplitude (TBI waveform) or phase (BR waveform) changes in the injected current. Typically circuitry for measuring TBI and BR waveforms are separate and both located on the circuit board 192.

For bio-impedance measurements, circuitry featuring one or more differential amplifiers connects to the electrodes 172A,B and generates a voltage that relates to the resistance (or impedance) through Ohms Law. Typically a bio-impedance circuit within the circuit board 192 measures TBI waveforms, which are separated into an AC waveform that features relatively high-frequency features (this waveform is typically called ΔZ(t)), and a DC waveform that features relatively low-frequency features (this waveform is typically called Z₀). This technique for measuring ΔZ(t) and Z₀ is described in detail in the following co-pending patent applications, the contents of which have been previously incorporated herein by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Feb. 19, 2014; and “BODY-WORN SENSOR FOR CHARACTERIZING PATIENTS WITH HEART FAILURE,” U.S. Ser. No. 14/145,253, filed Dec. 31, 2013, and ‘FLOORMAT PHYSIOLOGICAL SENSOR’, U.S.S.N FLOORMAT PHYSIOLOGICAL SENSOR (U.S. Ser. No. _{______}, Filed _{______}); COMBINED FLOORMAT AND BODY-WORN PHYSIOLOGICAL SENSORS (U.S. Ser. No. _{______}, Filed _{______}); HANDHELD PHYSIOLOGICAL SENSOR (U.S. Ser. No. _{______}, Filed _{______}); PHYSIOLOGICAL MONITORING SYSTEM FEATURING FLOORMAT AND HANDHELD SENSOR (U.S. Ser. No. _{______}, Filed _{______}); and PHYSIOLOGICAL MONITORING SYSTEM FEATURING FLOORMAT AND WIRED HANDHELD SENSOR.

With calibration the Z₀ waveform yields Fluid levels and changes therein, as concentrated in the user's lower extremities. Fluids are typically conductive, and thus Fluid levels vary inversely with impedance levels: an increase in Fluid level decreases impedance, while a decrease in Fluid level increases impedance. Typically changes in impedance parameters, which in turn indicate a corresponding change in Fluid level, are more relevant than absolute impedance levels.

A similar approach is used for bio-reactance and BR waveforms. However in this case, circuitry measures changes in phase corresponding to the injected current, as opposed to changes in amplitude used for bio-impedance. During a measurement, the phase difference between the injected currents and the detected currents is measured by the bio-reactance circuit and ultimately processed with the digital system on the circuit board to generate the BR waveform. The difference in phase is due to the current being slowed down by the capacitive properties of cell membranes within the conduction pathway. The baseline phase difference (Φa) is estimated from the DC component of the BR waveform. Φa is used to calculate tissue composition, described in more detail below. The AC component of the waveform can be used to track RR, SV, and CO as described above.

Bio-reactance, when combined with bio-impedance, can measure physiological parameters related to body composition (e.g. fat, muscle, and fluid in the user's body) and the progression of disease states. These parameters, like weight, may also be used to calibrate the SV measurement. Typically such a calibration is determined by conducting a large-scale clinical study using a known reference for SV and CO. More specifically, bio-impedance and bio-reactance measurements analyze the resistance and reactance of the user's tissue—along with biometric parameters such as height, weight and age—to generate accurate estimates of the composition of the tissue in the abdomen, chest, and arm. Such parameters may correlate with the size of the user's left ventricle and aorta, and can thus be used within V_{c}. Height, weight, and age, for example, can be input to the software application operating on the user's mobile device, and wirelessly transmitted to the Handheld Sensor for follow-on analysis (e.g., to calculate V_{c}).

Φa and Z₀ are then used to calculate the resistance (Z₀ cos(Φa)) and the reactance (Z₀ sin(Φa)) of the tissue in the abdomen, chest, and right arm. Resistance and reactance have been shown to be predictive of tissue composition. For example, fatty tissue is far more conductive than fat-free tissue. Therefore, a tissue's resistance is largely governed by the mass of the fat-free tissue present. This makes the inverse of a tissue's resistance a good estimator of that tissue's fat-free mass. Similarly, cell membranes have capacitive properties that cause phase changes in current that passes through the body. The greater the concentration of cells in the tissue, the greater the change in phase. When coupled with resistance, reactance can thus distinguish changes in fat from changes in fluid due to the differences in the cellularity of fat and extracellular fluid. Specifically, it has been shown that resistance and reactance—coupled with height, weight and age—can predict fat-free mass and body-fat mass as accurately as the “gold-standard” method—air displacement plethysmography. This is described in the following journal article, the contents of which are incorporated herein by reference: Body fat measurement by bioelectrical impedance and air displacement plethysmography: a cross-validation study to design bioelectrical impedance equations in Mexican adults; Nutrition Journal; 6: (2007). When fat-free mass, body-fat mass, and weight are measured, the root cause of changes in weight can be identified. Changes in fluid retention can signal the onset or reoccurrence of numerous medical conditions, such as CHF and ESRD. By measuring both reactance and resistance, both the Floormat and Handheld Sensor can distinguish changes in fluid retention from changes in tissue mass. This enables reliable tracking of this important parameter at home, on a daily basis. It also may improve the calculated accuracy of V_{c}, thereby improving the accuracy in calculating SV and CO

On its four corners the Floormat 100 features unique load cells 190A-D that collectively measure a voltage value that, following processing by an electrical circuit, can be converted by a linear algorithm into a weight value. More specifically, each load cell 190A-D includes a Wheatstone Bridge, which is an electrical circuit featuring one or more resistors having a resistance value that varies with strain caused by an applied weight. Each Wheatstone Bridge within the load cell connects through a 4-wire cable (not shown in the figure) to a differential amplifier located on the circuit board 192 within the Floormat 100. Separate differential amplifiers associated with each load cell amplify the voltage resulting from the load cell's Wheatstone Bridge. The resulting voltages pass to a summing amplifier on the circuit board 192 that adds and amplifies them to generate a single voltage that is then processed by a microprocessor on the circuit board to determine the user's weight.

Typically weight values, like the one described above, are measured from a single voltage value. However, the voltage values can also be sampled over time to generate a time-dependent voltage waveform that indicates a number of parameters associated with the user. For example, BCG pulses caused by small, heart-beat induced expansions in the user's feet can be mapped onto the waveforms, and thus used to further calculate parameters such as HR. BCG pulses are typically best measured using signal-processing techniques such as beatstacking, as described above. The BCG pulses can be collectively analyzed with PPG pulses to calculate a transit time, which relates inversely to BP as described in the above-referenced patent application entitled ‘FLOORMAT PHYSIOLOGICAL SENSOR’, the contents of which have been incorporated herein by reference. In other embodiments, time-dependent voltage waveforms measured by the load cells can be used to detect parameters such as balance and even progression of diseases such as Parkinson's disease. More specifically, a user that is swaying or undergoing related motions will generate a waveform that varies in amplitude over time; this may indicate a user with ‘bad’ balance. Likewise, a user that stands in a stable, unwavering manner on the Floormat will generate a waveform featuring relatively stable amplitude over time, thus indicating ‘good’ balance. In a similar manner, a user with Parkinson's disease typically undergoes small, rapid movements or tremors that will map onto the time-dependent voltage waveform. Analysis of frequency and amplitude components within the waveforms may indicate the progression of this disease.

On its top surface 102 the Floormat 100 also includes a ‘status bar’ 180 that is raised relative to the top surface 102 and includes a trio of status LEDs 182 indicating the Floormat's status, along with a pushbutton on/off switch 184. The status LEDs 182 indicate, for example, if the Floormat: i) is ready for the user to step on it; is making a measurement; is transmitting a measurement; or has completed a measurement. Other states of the Floormat, of course, can be indicated with the status LEDs 182. Each LED can emit a variety of colors, making it possible to indicate a large number of configurations to the user. As indicated by its name, the pushbutton on/off switch 184 turns the Floormat 100 on and off.

In a preferred embodiment, the Floormat 100 lacks a conventional display (e.g. an LCD). Instead it displays information on the software application running on the mobile device. In alternate embodiments the Floormat may include a conventional display.

### 3. Handheld Sensor

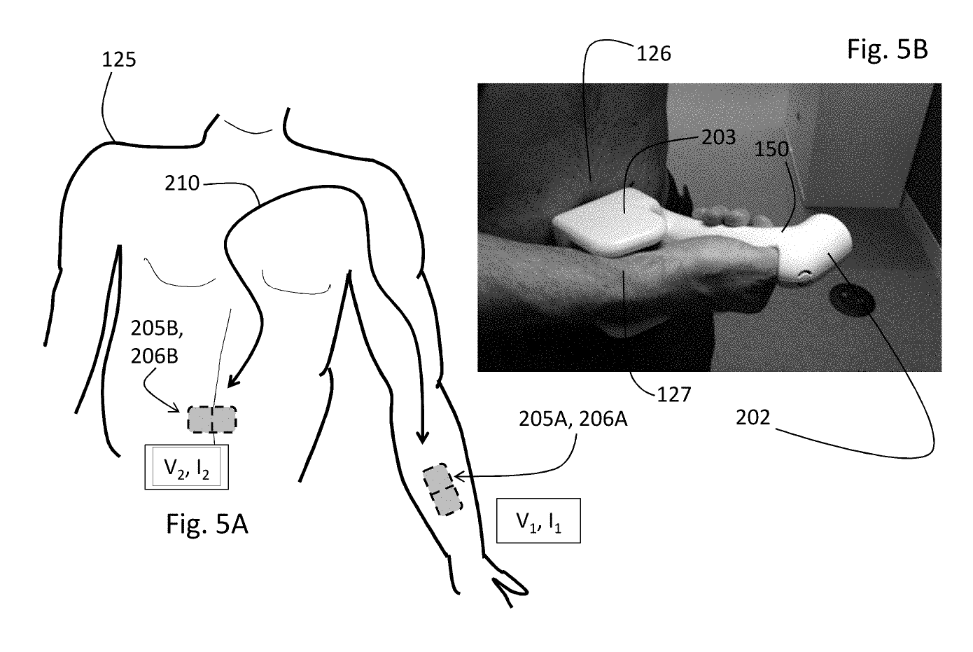

The Handheld Sensor 150 works in concert with the Floormat 100 and mobile device 125, as described above. Referring to FIGS. 5A and 5B, during a measurement the user stands on the Floormat, and simultaneously grasps the Handheld Sensor 150, inserts their wrist into a C-shaped opening 203 near the Sensor's bottom portion, and inserts their thumb into an opening near the Sensor's upper portion 202. The user then rests the Sensor's bottom portion against their belly with the top portion pointing outward, as shown in FIG. 5B. Ideally the user places the Sensor in the same position each day; the belly button is an ideal marker for placement.

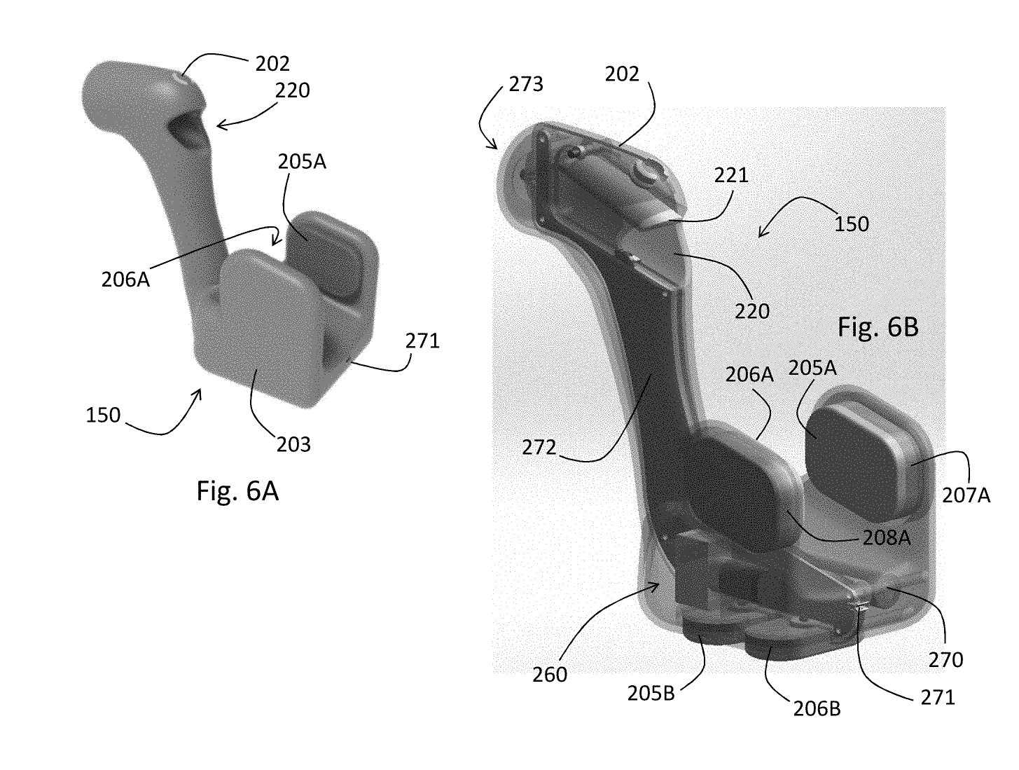

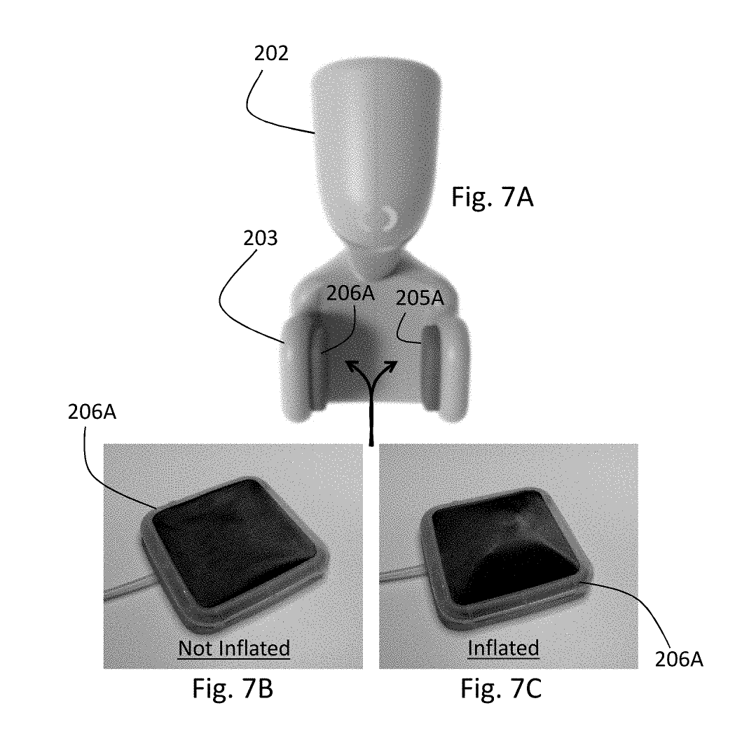

As described below in more detail with respect to FIGS. 6 and 7, both sides of the C-shaped opening include a first set of electrodes 205A, 206A that perform a function similar to that performed by the first set of electrodes 170A, 172A shown in FIGS. 3 and 4. The electrodes, which are more specifically described with respect to FIG. 7, contact the inside and outside portions of the user's wrist. A second set of electrodes 205B, 206B located on the bottom surface of the Sensor's bottom portion contact the user's belly when the Handheld Sensor 150 is placed as described above. In this way, the first set 205A, 206A and second set 205B, 206B of electrodes form a conduction path for performing a bio-impedance and/or bio-reactance measurement that extends from the user's belly, through their heart and lungs, and to their wrist, as indicated by arrow 210. Respiration parameters, fluids in the belly and thoracic cavity, and heartbeat-induced blood flow all modulate electrical current injected by the electrodes to form TBI and BR waveforms. Algorithms operating on the Handheld Sensor process these waveforms to determine Fluids, RR SV, and ultimately CO as described in detail below. Moreover, use of the Handheld Sensor in this manner ensures relatively consistent placement on a day-to-day basis, thereby minimizing placement-related errors in the measurement.

The Floormat sensor is optimized for measuring Z₀, and from this parameter Fluids, from the lower extremities (e.g. legs) of the user's body. Complementing this is the Handheld Sensor's measurement of TFC from the upper portion of the user's body. Moreover, cardiac-related processes that modulate the AC portion of the bio-impedance waveform (e.g. ΔZ(t)) are typically easier to measure with the Handheld Sensor. As shown in FIG. 5A, when used as described above on a user 125, the Handheld Sensor 150 injects current (indicated by I1, I2) and detects voltage (indicated by V1, V2) over the conduction pathway 210 that extends from the user's wrist to their belly button. Physiological processes that take place within this path may modulate the TBI waveform. For example, respiratory effort (i.e. breathing) changes the capacitance of the chest, thus imparting a series of low-frequency undulations (typically 5-30 undulations/minute) on the ΔZ(t) waveform. The Handheld Sensor's digital system processes these oscillations to determine RR.

Blood is a decent electrical conductor, and thus blood pumped by the heart's left ventricle into the aorta modulates impedance in the thoracic cavity (as well as other regions spanned by the conduction pathway 210, e.g. the brachial artery located in the user's bicep). These modulations manifest as heartbeat-induced cardiac pulses on the ΔZ(t) waveform. They can be processed to determine SV as described in detail in the following co-pending patent applications, the contents of which have been previously incorporated by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Feb. 19, 2014; and “BODY-WORN SENSOR FOR CHARACTERIZING PATIENTS WITH HEART FAILURE,” U.S. Ser. No. 14/145,253, filed Dec. 31, 2013, and ‘FLOORMAT PHYSIOLOGICAL SENSOR’, U.S.S.N FLOORMAT PHYSIOLOGICAL SENSOR (U.S. Ser. No. _{______}, Filed _{______}); COMBINED FLOORMAT AND BODY-WORN PHYSIOLOGICAL SENSORS (U.S. Ser. No. _{______}, Filed _{______}); HANDHELD PHYSIOLOGICAL SENSOR (U.S. Ser. No. _{______}, Filed _{______}); PHYSIOLOGICAL MONITORING SYSTEM FEATURING FLOORMAT AND HANDHELD SENSOR (U.S. Ser. No. _{______}, Filed _{______}); and PHYSIOLOGICAL MONITORING SYSTEM FEATURING FLOORMAT AND WIRED HANDHELD SENSOR. The Handheld Sensor determines CO, which is the product of SV and HR, using a simple calculation.

Fluids (e.g. TFC) also conduct the injected current. Thus fluids that accumulate in the thoracic cavity affect the impedance within the conduction pathway 210 in a low-frequency (i.e. slowly changing) manner, and can be detected by processing the Z₀ waveform. Typically the Z₀ waveform features an average value of between about 10-30 Ohms, with 10 Ohms indicating relatively low impedance and thus high fluid content (e.g. the user is ‘wet’), and 30 Ohms indicating a relatively high impedance and thus low fluid content (e.g. the user is ‘dry’). Time-dependent changes in the average value of Z₀ can indicate that the user's fluid level is either increasing or decreasing. An increase in fluid level, for example, may indicate the onset of CHF.

The same electrodes used to measure TBI and BR waveforms also measure ECG waveforms, as described below. The relatively long conduction path 210 ensures ECG waveforms are measured using bioelectric signals having a large potential difference; this typically yields waveforms with relatively high signal-to-noise ratios. However, in embodiments, additional electrodes may be employed to enable a “driven right-leg” circuit to reduce noise in the ECG waveform. Such circuits are know in the art for reducing noise (typically at 50 or 60 Hz) due to the common mode. These additional electrodes may be located adjacent to existing electrodes, on the padding around the optical sensor, or along the neck of the handheld device. The ECG waveform features heartbeat-induced pulses that, informally, mark the beginning of the cardiac cycle. Typically the pulses include a sharp feature, called a QRS complex, which indicates electrical activity in the heart. The time separating neighboring QRS complexes is inversely related to the user's HR. Typically HR is calculated from a collection of QRS complexes spanning a short period of time, e.g. 30 seconds. The variation in heart rate determined during this period is the HRV, which is known to relate to cardiac function.

From these waveforms an algorithm can also determine other parameters that may be extracted from the ECG waveform, such as the presence of T-waves, P-waves, and elevation of the ST segment. Other ECG analysis techniques known in the art are also within the scope of the invention. Such analysis techniques are described in the following co-pending patent applications, the contents of which are incorporated herein by reference: ‘INTERNET-BASED SYSTEM FOR EVALUATING ECG WAVEFORMS TO DETERMINE THE PRESENCE OF P-MITRALE AND P-PULMONALE’, U.S. Ser. No. 14/048,701, Filed Oct. 8, 2013.

FIGS. 6A,B and 7A-C show the Handheld Sensor 150 in more detail. The Sensor 150 measures PPG waveforms, and from these SpO2 and HR, using optical components (red/infrared LEDs and a photodetector, similar to those described above for the Floormat) housed in an opening 220 that receives the user's thumb. A spring-loaded finger-clip sensor 221, similar to the toe-clip sensor 200 shown in FIGS. 3 and 4, applies a light pressure to the thumb to facilitate the optical measurement. Signal-processing techniques, circuitry, and algorithms used to make these measurements are similar to those used for the Floormat, as described above.

The same PPG waveforms used to measure SpO2 can also be utilized to measure BP, and specifically SYS. To make this measurement the first set of electrodes 205A, 206A are typically formed from a stretchable, conductive fabric, as shown in more detail in FIGS. 7B and 7C. For each electrode 205A, 206A the fabric is stretched over an inflatable bladder 207A, 208A that connects to a pneumatic system 260 that includes a microprocessor-controlled pump and valve. During a measurement, the pneumatic system 260 slowly inflates the bladders 207A, 208A, thus pressing the electrodes 205A, 206A on each side of the user's wrist. This gradually occludes the radial and ulnar arteries that supply blood to the user's thumb, which is inserted into the opening 220 that houses the optical system measuring PPG waveforms. As pressure is applied, heartbeat-induced pulses in the PPG waveforms gradually decrease in amplitude. The pulses are completely eliminated when the applied pressure equals the user's SYS. Thus, monitoring the pulses' disappearance with an algorithm yields SYS.

Simultaneously, heartbeat-induced pulsations from the radial and ulnar arteries couple into the bladders 207A, 208A, where they can be measured using electrical circuitry that includes filters and amplifiers designed to measure pressure waveforms. The waveforms can be processed to isolate AC components (which include the pulsations) and DC components (which can be analyzed to determine the applied pressure). Collective analysis of the AC and DC waveforms yields a bell-shaped curve when the amplitude of each pressure pulsation is plotted against the pressure applied. The digital system processes the bell-shaped curve to determine blood pressure according to the well-known technique of oscillometry. Such a technique is known in the art. Typically an algorithm determines the maximum value of the bell-shaped curve, which yields the user's MAP. SYS can be determined as described above from the PPG waveform, or alternatively from applied pressures that yield well-defined amplitudes on the high-pressure side of MAP. More specifically, SYS typically corresponds to the applied pressure that yields a pulse amplitude on the high-pressure side of MAP that, when divided by the pulse amplitude corresponding to MAP, has a ratio of about 0.4. Similarly, DIA can be determined from the same bell-shaped curve. The pressure typically corresponds to the applied pressure that yields a pulse amplitude on the low-pressure side of MAP that, when divided by the pulse amplitude corresponding to MAP, has a ratio of 0.6. Other ratios can also be used to calculate SYS and DIA according to oscillometry

In embodiments, algorithms running on the microprocessor may be used to compensate for blood pressure differences between the wrist and bicep, which are commonly caused by hydrostatic forces within the body. Such algorithms are known in the art and typically depend on the user's height. To some extent these algorithms are simplified by the requirement that the user hold the Handheld Sensor in the same location each day (the belly) when making a measurement.

The Handheld Sensor 150 also includes an infrared temperature sensor 273 in its upper portion 202 for measuring TEMP. For this measurement, the user grasps the Sensor 150 in a manner similar to that shown in FIG. 5B, and holds it up near their head so that the temperature sensor 272 points into the ear. Accelerometers within the Sensor detect motions required to position the temperature sensor 272 as such, and are analyzed by the microprocessor to activate the measurement of TEMP. Measurements of TEMP typically only take a few moments, and are done using standard techniques within the art. For example, for this measurement, the temperature sensor 272 detects infrared radiation (e.g. blackbody radiation) emitted from inside the ear, which it then converts to a temperature using techniques known in the art. Typically the temperature sensor 272 is a fully digital system, meaning it receives the infrared radiation with an internal photodetector and, using an internal digital system, converts this to a temperature value that it sends through a serial interface (e.g. one based on a conventional UART or I2C interface) to a microprocessor for follow-on processing.

The Handheld Sensor 150 also includes other electrical/mechanical components, such as a pair of rechargeable batteries 270 that power all the above-described components, a micro-USB port 271 for charging the batteries and transferring data from the Sensor 150 to, e.g., a remote computer, and a circuit board 272 that includes all the Sensor's electronic components. The circuit board 272 includes, for example, a microprocessor that runs computer code for operating all the algorithms associated with the measurements described above, along with all discrete electrical components (e.g. resistors, capacitors, amplifiers) for analog and digital circuits used to make the Sensor's various measurements.

Both the Floormat and Handheld Sensor may include a vibrating component indicating when a measurement is complete. For example, the user typically holds the Handheld Sensor near their belly for about 30 seconds, after which an internal indicator (e.g., a buzzer coupled with a status LED) indicates that the measurement is complete. Once this occurs, an internal Bluetooth® transmitter in the Sensor transmits numerical and waveform information to the user's mobile device, which then forwards it to a web-based system. There, a clinician, the user, family member, etc. can review the information.

The accelerometers described above are preferably included within both the Handheld Sensor and Floormat to detect motion of the user. This information can be used, for example, to improve measurement quality by selectively detecting an ideal measurement period when motion is minimized. Accelerometers can also be used to detect the user's motion and thus initiate specific measurements, such as measurement of TEMP as described above, and also measurements performed by the Floormat. This approach, for example, would obviate the pushbutton on/off switch (component 184 in FIGS. 3 and 4) described above.

### 4. Other Measurements—Pulse Transit Time

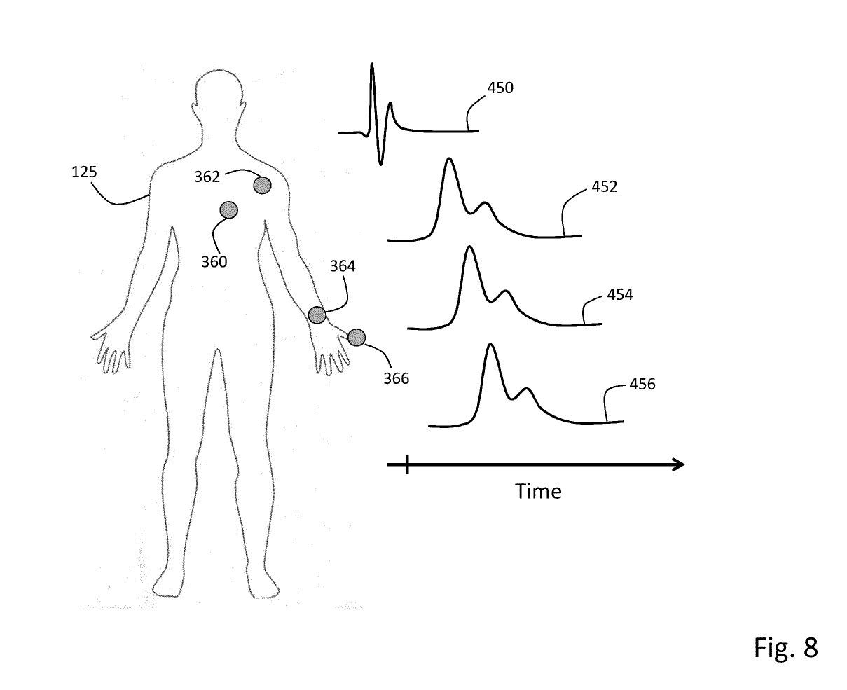

Referring to FIG. 8, the Handheld Sensor measures from a user 125 heartbeat-induced pulsatile components from the following waveforms: ECG 450, TBI 452, oscillometric 454, and PPG 456. As indicated in the figure, the Handheld Sensor samples pulsatile components in these waveforms along different portions of the user's body, with each portion separated from the source of the pulsatile components—the user's heart—by a sequentially increasing distance. For example, optics (LED, photodetector) within either the Floormat and/or Handheld Sensor measure pulsatile components in the PPG waveform 456, sampled from arteries within the user's thumb 366. The inflatable bladders in the C-shaped portion, coupled with pressure-measuring electronics, sense pulsatile components from the oscillometric waveform 454 measured from the user's wrist 364. Stretchable cloth electrodes and the bio-impedance (and optionally bio-reactance) circuits measure pulsatile components in the ΔZ(t) waveform 452, which primarily senses blood flow from the heart's left ventricle into the aorta 362. And the QRS complex of the ECG waveform 450 is a pulsatile component that indicates initial electrical activity in the user's heart 360 and, informally, marks the beginning of the cardiac cycle.

Thus detection and analysis of each of the above-described pulsatile components indicates blood flow through the user's body. More specifically, the digital system in the handheld component can analyze the pulsatile components to determine parameters such as pulse arrival time (PAT), pulse transit time (PTT), and vascular transit time (VTT). Such transit times can be used, for example, to calculate blood pressure, e.g. SYS, DIA, and MAP. This methodology is described in more detail in the following co-pending patent applications, the contents of which have been previously incorporated herein by reference: “NECK-WORN PHYSIOLOGICAL MONITOR,” U.S. Ser. No. 62/049,279, filed Sep. 11, 2014; “NECKLACE-SHAPED PHYSIOLOGICAL MONITOR,” U.S. Ser. No. 14/184,616, filed Feb. 19, 2014; and “BODY-WORN SENSOR FOR CHARACTERIZING PATIENTS WITH HEART FAILURE,” U.S. Ser. No. 14/145,253, filed Dec. 31, 2013, and ‘FLOORMAT PHYSIOLOGICAL SENSOR’, U.S.S.N FLOORMAT PHYSIOLOGICAL SENSOR (U.S. Ser. No. _{______}, Filed _{______}); COMBINED FLOORMAT AND BODY-WORN PHYSIOLOGICAL SENSORS (U.S. Ser. No. _{______}, Filed _{______}); HANDHELD PHYSIOLOGICAL SENSOR (U.S. Ser. No. _{______}, Filed _{______}); PHYSIOLOGICAL MONITORING SYSTEM FEATURING FLOORMAT AND HANDHELD SENSOR (U.S. Ser. No. _{______}, Filed _{______}); and PHYSIOLOGICAL MONITORING SYSTEM FEATURING FLOORMAT AND WIRED HANDHELD SENSOR.

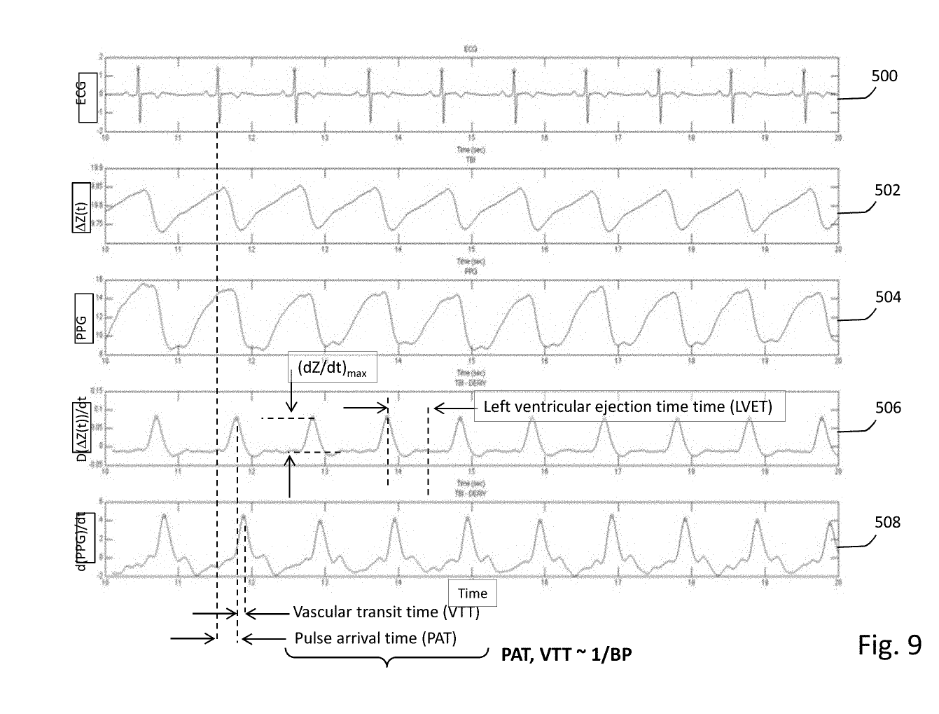

To summarize, FIG. 9 shows the following time-dependent waveforms, as measured by the Floormat and/or Handheld Sensor: ECG (plot 500), ΔZ(t) (plot 502), PPG (plot 504), d(ΔZ(t))/dt (plot 506), and d(PPG)/dt (plot 508). As shown in plots 500 and 502, individual heartbeats produce time-dependent pulses in both the ECG and ΔZ(t) waveforms. As is clear from the data, pulses in the ECG waveform precede those in the ΔZ(t) waveform. The ECG pulses—each featuring a sharp, rapidly rising QRS complex—mark the beginning of the cardiac cycle.

ΔZ(t) pulses follow the QRS complex by about 100 ms and indicate blood flow through arteries in the region of the body where the cloth electrodes make contact with the skin. During a heartbeat, blood flows from the user's left ventricle into the aorta; the volume of blood that leaves the ventricle is the SV. Blood flow periodically enlarges this vessel, which is typically very flexible, and also temporarily aligns blood cells (called erythrocytes) from their normally random orientation. Both the temporary enlargement of the vessel and alignment of the erythrocytes improves blood-based electrical conduction, thus decreasing the electrical impedance as measured with ΔZ(t). The d(ΔZ(t))/dt waveform (plot 506) shown in FIG. 9 is a first mathematical derivative of the raw ΔZ(t) waveform, meaning its peak represents the point of maximum impedance change.

A variety of time-dependent parameters can be extracted from the ECG and TBI waveforms. For example, as noted above, it is well know that HR can be determined from the time separating neighboring ECG QRS complexes. Likewise, left ventricular ejection time (LVET) can be measured directly from the derivative of pulses within the ΔZ(t) waveform, and is determined from the onset of the derivatized pulse to the first positive-going zero crossing. Also measured from the derivatized pulses in the ΔZ(t) waveform is (d□ΔZ(t))/dt)ₘₐₓ, which is a parameter used to calculate SV as described above.

The time difference between the ECG QRS complex and the peak of the derivatized ΔZ(t) waveform represents a pulse arrival time PAT, as indicated in FIG. 9. This value can be calculated from other fiducial points, including, in particular, locations on the ΔZ(t) waveform such as the base, midway point, or maximum of the heartbeat-induced pulse. Typically, the maximum of the derivatized waveform is used to calculate PAT, as it is relatively easy to develop a software beat-picking algorithm that finds this fiducial point.

PAT correlates inversely to SYS, DIA, and MAP, which can be calculated as described in the above-referenced patent applications using user-specific slopes for SYS and DIA, measured during a calibration measurement. (Such a measurement can, for example, be performed with the inflatable bladders and optical systems described above.) Without the calibration, PAT only indicates relative changes in SYS, DIA, and MAP. The calibration yields both the user's immediate values of these parameters. Multiple values of PAT and blood pressure can be collected and analyzed to determine user-specific slopes, which relate changes in PAT with changes in SYS, DIA, and MAP. The user-specific slopes can also be determined using pre-determined values from a clinical study, and then combining these measurements with biometric parameters (e.g. age, gender, height, weight) collected during the clinical study.

In embodiments of the Handheld Sensor, waveforms like those shown in FIG. 9 can be processed to determine transit times such as PAT and VTT. The Floormat and/or Handheld Sensor can use these parameters, combined with a calibration determined as described above, to calculate blood pressure without a mechanism that applies pressure, e.g. the inflatable bladders described above. Typically PAT and SYS correlate better than PAT and DIA.

PP can be used to calculate DIA from SYS, and can be estimated from either the absolute value of SV, SV modified by another property (e.g. LVET), or the change in SV. In the first method, a simple linear model is used to process SV (or, alternatively, SV×LVET) and convert it into PP. The model uses the instant values of PP and SV, determined as described above from a calibration measurement, along with a slope that relates PP and SV (or SV×LVET) to each other. The slope can be estimated from a universal model that, in turn, is determined using a population study.

Alternatively, a slope tailored to the individual user can be used. Such a slope can be selected, for example, using biometric parameters characterizing the user as described above.

Here, PP/SV slopes corresponding to such biometric parameters are determined from a large population study and then stored in computer memory on the Floormat and/or Handheld Sensor. When a device is assigned to a user, their biometric data is entered into the system, e.g. using a GUI operating on a mobile device, that transmits the data to the Floormat and/or Handheld Sensor via Bluetooth®. Then, an algorithm processes the data and selects a user-specific slope. Calculation of PP from SV is explained in the following reference, the contents of which are incorporated herein by reference: “Pressure-Flow Studies in Man. An Evaluation of the Duration of the Phases of Systole,” Harley et al., Journal of Clinical Investigation, Vol. 48, p. 895-905, 1969. As explained in this reference, the relationship between PP and SV for a given user typically has a correlation coefficient r that is greater than 0.9, which indicates excellent agreement between these two properties. Similarly, in the above-mentioned reference, SV is shown to correlate with the product of PP and LVET, with most users showing an r value of greater than 0.93 and the pooled correlation value (i.e., the correlation value for all subjects) being 0.77. This last value indicates that a single linear relationship between PP, SV, and LVET may hold for all users.

More preferably, PP is determined from SV using relative changes in these values. Typically, the relationship between the change in SV and change in PP is relatively constant across all subjects. Thus, similar to the case for PP, SV, and LVET, a single, linear relationship can be used to relate changes in SV and changes in PP. Such a relationship is described in the following reference, the contents of which are incorporated herein by reference: “Pulse pressure variation and stroke volume variation during increased intra-abdominal pressure: an experimental study,” Didier et al., Critical Care, Vol. 15:R33, p. 1-9, 2011. Here, the relationship between PP variation and SV variation for 67 subjects displayed a linear correlation of r=0.93, which is an extremely high value for pooled results that indicates a single, linear relationship may hold for all users.

From such a relationship, PP can be determined from the impedance-based SV measurement, and SYS can be determined from PAT. DIA can then be calculated from SYS and PP.

Another parameter, VTT, can be determined from pulsatile components in the ΔZ(t) (or d(ΔZ(t))/dt) waveform and the PPG (or d(PPG)/dt) waveform. FIG. 9 shows in more detail how VTT is determined. It can be used in place of PAT to determine blood pressure, as described above. Using VTT instead of PAT in this capacity offers certain advantages, namely, lack of signal artifacts such as pre-injection period (PEP) and isovolumic contraction time (ICT), which contribute components to the PAT value but which are not necessarily sensitive to or indicative of blood pressure.

In general, the overarching purpose of a system that combines the Floormat and Handheld Sensor according to the invention, as described above, is to make daily measurements of a wide range of physiological parameters that, in turn, can be analyzed to diagnose specific disease states. Use of a single system, as opposed to multiple devices, can simplify operation and reduce the time required to measure the above-mentioned parameters. This, in turn, may increase the user's compliance, as it is well established that daily use of devices that measure physiological parameters typically improves as the time and complexity required for such devices decreases.

By consistently collecting physiological information on a daily basis, the combined Floormat and Handheld Sensor can calculate trends in the information. Such trends may indicate the progression of certain disease states in a manner that is improved relative to one-time measurements of certain parameters. For example, a value of fluids corresponding to 15 Ohms, or an SV corresponding to 75 mL, has little value taken in isolation. But if these parameters decrease by 20% over a period of a few days, it can indicate that the user's heart is pumping blood in a less efficient manner (as indicated by the SV), which in turn decreases perfusion of their kidneys and causes them to retain more fluids (as indicated by the fluid level).

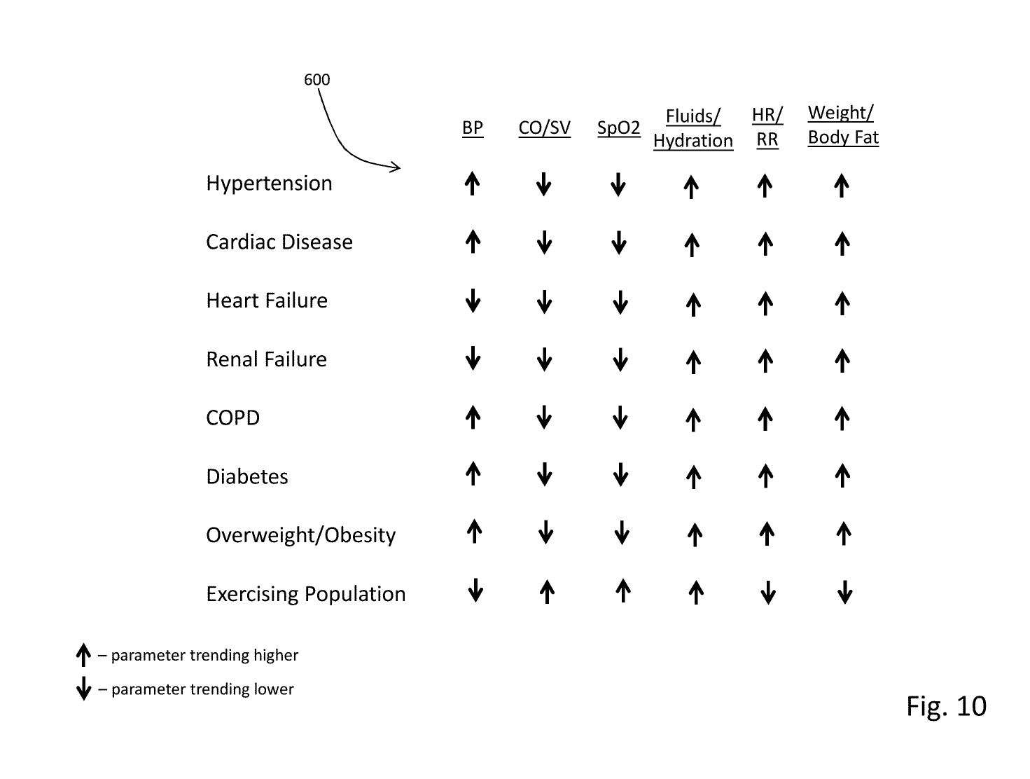

In this regard, FIG. 10 shows, for example, a table 600 indicating how trends in different physiological parameters can be used to diagnose disease states such as hypertension, cardiac disease, HF (including CHF), renal failure (including ESRD), COPD, diabetes, and obesity. In addition, the table 600 indicates how such trends may show beneficial progress to a population actively involved in exercise.

In other embodiments, the Floormat described above can integrate with a ‘patch’ that directly adheres to a portion of a patient's body, or a ‘necklace’ that drapes around the patient's neck. The patch would be similar in form to the necklace's base, although it may take on other shapes and form factors. It would include most or all of the same sensors (e.g. sensors for measuring ECG, TBI, and PPG waveforms) and computing systems (e.g. microprocessors operating algorithms for processing these waveforms to determine parameters such as HR, HRV, RR, BP, SpO2, TEMP, CO, SV, fluids) as the base of the necklace. However unlike the system described above, the battery to power the patch would be located in or proximal to the base, as opposed to the strands in the case of the necklace. Also, in embodiments, the patch would include a mechanism such as a button or tab functioning as an on/off switch. Alternatively, the patch would power on when sensors therein (e.g. ECG or temperature sensors) detect that it is attached to a patient.

In typical embodiments, the patch includes a reusable electronics module (shaped, e.g., like the base of the necklace) that snaps into a disposable component that includes electrodes similar to those described above. The patch may also include openings for optical and temperature sensors as described above. In embodiments, for example, the disposable component can be a single disposable component that receives the reusable electronics module. In other embodiments, the reusable electronics module can include a reusable electrode (made, e.g., from a conductive fabric or elastomer), and the disposable component can be a simple adhesive component that adheres the reusable electrode to the patient.

In preferred embodiments the patch is worn on the chest, and thus includes both rigid and flexible circuitry, as described above. In other embodiments, the patch only includes rigid circuitry and is designed to fit on other portions of the patient's body that is more flat (e.g. the shoulder).

In embodiments, for example, the system described above can calibrate the patch or necklace for future use. For example, the Floormat can determine a patient-specific relationship between transit time and blood pressure, along with initial values of SYS, DIA, and MAP. Collectively these parameters represent a cuff-based calibration for blood pressure, which can be used by the patch or necklace for cuffless measurements of blood pressure. In other embodiments, the Floormat can measure a full-body impedance measurement and weight. These parameters can be wirelessly transmitted to the necklace or patch, where they are used with their impedance measurement to estimate full-body impedance (e.g. during a dialysis session). Additionally, during the dialysis session, the necklace or patch can use the values of full-body impedance and weight to estimate a progression towards the patient's dry weight.

These and other embodiments of the invention are deemed to be within the scope of the following claims.

What is claimed is:

1. A system for monitoring a pulse transit time from a patient, comprising:
a floormat sensor configured to rest on a substantially horizontal surface, the floormat sensor comprising:
an optical system connected to a top surface of the floormat sensor and comprising an optical system with at least one light source and a photodetector configured to measure a time-dependent photoplethysmogram waveform from the patient while they are standing on the floormat sensor;
a handheld sensor configured to be held in the patient's hand and comprising:
a physiological monitoring system configured to measure at least one time-dependent physiological waveform from a location near a hand of the patient while they are standing on the floormat sensor; and
a processing system configured to receive the time-dependent photoplethysmogram waveform from the floormat sensor, and the at least one time-dependent physiological waveform from the handheld sensor, and then collectively process the waveforms to determine the pulse transit time.

2. The system of claim 1, wherein the processing system comprises computer code configured to: 1) calculate a mathematical derivative of the photoplethysmogram waveform or a version thereof to determine a set of derivative values; and 2) determine a local maximum of the set of derivative values to determine a first pulsatile component.

3. The system of claim 1, wherein the time-dependent physiological waveform is an ECG waveform.

4. The system of claim 3, wherein the processing system comprises computer code configured to determine a QRS complex in the ECG waveform to determine a second pulsatile component.

5. The system of claim 4, wherein the processing system comprises computer code configured to determine an R point in the QRS complex in the ECG waveform to determine the second pulsatile component.

6. The system of claim 4, wherein the processing system comprises computer code configured to determine a Q point in the QRS complex in the ECG waveform to determine the second pulsatile component.

7. The system of claim 4, wherein the processing system determines a temporal difference between the first and second pulsatile components to determine the pulse transit time.

8. The system of claim 7, wherein the processing system further processes the pulse transit time to calculate a blood pressure value.

9. The system of claim 5, wherein the processing system determines a temporal difference between the first and second pulsatile components to determine the pulse transit time.

10. The system of claim 9, wherein the processing system further processes the pulse transit time to calculate a blood pressure value.

11. The system of claim 6, wherein the processing system determines a temporal difference between the first and second pulsatile components to determine the pulse transit time.

12. The system of claim 11, wherein the processing system further processes the pulse transit time to calculate a blood pressure value.

13. The system of claim 1, wherein the time-dependent physiological waveform is a second photoplethysmogram waveform or a version thereof.

14. The system of claim 13, wherein the processing system comprises computer code configured to determine a maximum value of the photoplethysmogram waveform to determine a second pulsatile component.

15. The system of claim 13, wherein the processing system comprises computer code configured to determine a maximum value of a derivative of the photoplethysmogram waveform to determine a second pulsatile component.

16. The system of claim 14, wherein the processing system determines a temporal difference between the first and second pulsatile components to determine the pulse transit time.

17. The system of claim 16, wherein the processing system further processes the pulse transit time to calculate a blood pressure value.

18. The system of claim 15, wherein the processing system determines a temporal difference between the first and second pulsatile components to determine the pulse transit time.

19. The system of claim 18, wherein the processing system further processes the pulse transit time to calculate a blood pressure value.

20. The system of claim 1, wherein the time-dependent physiological waveform is an impedance waveform or a version thereof.

21. The system of claim 20, wherein the processing system comprises computer code configured to determine a maximum value of the impedance waveform to determine a second pulsatile component.

22. The system of claim 20, wherein the processing system comprises computer code configured to determine a maximum value of a derivative of the impedance waveform to determine a second pulsatile component.

23. The system of claim 21, wherein the processing system determines a temporal difference between the first and second pulsatile components to determine the pulse transit time.

24. The system of claim 23, wherein the processing system further processes the pulse transit time to calculate a blood pressure value.

25. The system of claim 22, wherein the processing system determines a temporal difference between the first and second pulsatile components to determine the pulse transit time.

26. The system of claim 25, wherein the processing system further processes the pulse transit time to calculate a blood pressure value.
